(19) United States
(12) Patent Application Publication
(10) Pub. No.: US 2005/0282456 A1
(43) Pub. Date: Dec. 22, 2005

(54) Laminate comprising a polyhydroxyalkanoate copolymer

(75) Inventors: Jean Jianqun Zhao, Montgomery, OH (US); Kathleen Bridget Jividen, Turtlecreek Twp, OH (US); Gary Wayne Gilbertson, Liberty Twp, OH (US); Brian Francis Gray, Wyoming, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/870,763

(22) Filed: Jun. 17, 2004

(51) Int. Cl. B32B 27/12; B32B 27/36; A61F 13/20

(52) U.S. Cl. 442/394; 442/395; 442/414; 604/358; 604/366

(57) ABSTRACT

A laminate that is useful as a flushable, fluid impervious backsheet for flushable absorbent articles. The laminate includes a polyhydroxyalkanoate copolymer and a nonwoven. The flushable absorbent article includes a fluid permeable topsheet, absorbent core, and a flushable fluid impervious backsheet including the same laminate.

### FIELD OF INVENTION

The present invention relates to flushable and biodegradable laminates that comprise a polyhydroxyalkanoate copolymer. The laminates are useful in disposable absorbent articles, particularly in feminine hygiene products such as sanitary napkins, pantiliners and interlabial devices. The laminates are especially useful as the backsheet and/or the outer cover of flushable absorbent articles.

### BACKGROUND OF THE INVENTION

Disposable absorbent articles such as feminine hygiene products, diapers, training pants, and adult incontinence products, offer great convenience and are widely used by consumers. The typical disposal absorbent article usually comprises a topsheet, a backsheet, and an absorbent core positioned between the backsheet and the topsheet. Depending on the type of use involved, disposable articles can be subjected to one or more insults from aqueous liquids such as water, urine, menses or blood. As a result, the backsheet materials of these products are typically made of liquid impermeable materials, such as polypropylene or polyethylene films, that exhibit sufficient strength and handling capability so that the disposable absorbent article retains its integrity during use by the wearer and does not allow leakage of the liquid from the product.

The popularity of disposable absorbent articles has created a great concern regarding their disposal. Typical disposal methods such as incineration or landfill are costly and problematic to the environment. Therefore, there is a need for absorbent products that can be easily and cheaply disposed of without creating additional problems. An alternative disposal method has been proposed, which involves flushing the article down the conventional toilet and through the plumbing system without creating blockage. More importantly, the suitable materials should degrade anaerobically in the sewage or septic system such that there is no accumulation of large chunks of the materials in the system. The last requirement is known to be the most challenging.

Water-soluble materials, such as polyethylene oxide and polyvinyl alcohol, are known to quickly lose their integrity when exposed to a large quantity of water, thus they do not block the toilet and plumbing system, nor accumulate in the sewage or septic system. WO 96/20831A1 publication published by Larson, et al on Jul. 11, 1996, discloses a film that is substantially dispersed when contacted with an excess amount of water and can be used as a backsheet in a disposable absorbent article. This film comprises a water dispersible layer and a barrier layer. The water dispersible layer comprises a water dispersible material such as polyethylene oxide or polyvinyl alcohol and optionally a non-water dispersible polymer to improve the wet strength and handling properties of this layer. The barrier layer is typically prepared from a polymer that forms a film that is liquid impermeable such as acrylic acid copolymers and biodegradable aliphatic polyesters, and optionally a water dispersible polymer in order to improve the decomposition properties of the barrier layer. U.S. Pat. No. 6,514,602 issued to Zhao, et al. on Feb. 4, 2003, disclosed a water flushable and biodegradable multi-layer backsheet film. The film comprises a polymer blend of water dispersible materials such as polyethylene oxide or polyvinyl alcohol and biodegradable materials such as aliphatic polyesters, aliphatic-aromatic polyesters and polyester amides in each layer. The ratio of the blends in each layer is different in order to balance the barrier property (in-use integrity) and flushibility. However, these backsheets tend to be overly sensitive to moisture and to deform or grow mold in hot and humid conditions during shipping, storage and in usage.

Other materials, such as poly(lactic acid) polymers, polycaprolactones disclosed in U.S. Pat. No. 5,391,423 issued to Wnuk et al. on Feb. 21, 1995, aliphatic polyalkylene succinate polymers, polyalkylene succinate adipate copolymers or mixtures thereof, such as disclosed in U.S. Pat. No. 5,849,401 issued to El-Afandi et al. on Dec. 15, 1998 and U.S. Pat. No. 5,910,545 issued to Tsai, et al. on issued Jun. 8, 1999, aliphatic-aromatic copolyesters disclosed in U.S. Pat. No. 5,292,783 issued to Buchanan et, al. on Mar. 8, 1994, aliphatic polyesteramides disclosed in U.S. Pat. No. 5,644,020 issued to Timmermann et al. on Jul. 1, 1997, are known to be biodegradable under an aerobic conditions, yet generally fail to degrade significantly under anaerobic conditions within a reasonable amount of time such that no accumulation occurs in the system.

As opposed to the above materials, the polyhydroxyalkanoate copolymers disclosed in U.S. Pat. No. 5,489,470 issued to Isao Noda on Feb. 6, 1996 and U.S. Pat. No. 5,498,692 issued to Isao Noda on Mar. 12, 1996 are not only aerobically degradable but also anaerobically degradable. Backsheet films made of these copolymers are fluid impermeable, provide good barrier properties and degrade rapidly after flushing down the toilet. The degradation rate is comparable to natural materials, such as cellulose, starch, etc. However, it is well known that the melt processing of polyhydroxyalkanoate copolymers is quite problematic. One reason melt processing is problematic is that the melting point (Tm range from 80° C. to 160° C.) and the thermal degradation point (Td˜160° C.) of the polyhydroxyalkanoate copolymers are very close. Thus, the processing window is very narrow. The other reason melt processing is problematic is that polyhydroxyalkanoate copolymers are semi crystalline polymers. The rate of crystallization is noticeably slower than traditional commercial polymers, making their melt processing either impossible or cost-prohibitive on existing converting equipment, for they lack sufficient structural integrity or they remain substantially tacky, or both, after they are cooled down from the melt, and remain as such until sufficient crystallization sets in. Residual tack typically leads to the material sticking to itself or to processing equipment, or both, and thereby can restrict the speed at which a polymeric product is produced or prevent the product from being collected in a form of suitable quality.

The present invention overcomes the historical problems of the hot melt process of polyhydroxyalknoate copolymers. The present laminate and flushable, fluid impervious backsheet can be melt processed on conventional process equipment at typical manufacturing speeds. The present laminate and flushable, fluid impervious backsheet are fluid impervious and dispersible after being flushed down the toilet.

### SUMMARY OF THE INVENTION

The present invention relates to a laminate that is useful as a flushable, fluid impervious backsheet for flushable absorbent articles. The laminate comprises a polyhydroxyalkanoate copolymer and a nonwoven. The flushable absorbent article comprises a fluid permeable topsheet, absorbent core, and a flushable fluid impervious backsheet comprising the same laminate.

### BRIEF DESCRIPTION OF THE DRAWINGS

FIG. 1 shows a partial cut away plan view of an absorbent article comprising the flushable, fluid impervious backsheet of the present invention.

FIG. 2 shows a transverse cross-section of an absorbent article comprising the flushable, fluid impervious backsheet of the present invention.

FIG. 3 shows a cross sectional view of the laminate of the present invention.

### DETAILED DESCRIPTION OF THE INVENTION

The term “absorbent articles” refers to tampons, sanitary napkins, sanitary panties, interlabial devices, pantiliners, infant diapers, adult incontinence diapers, bandages, surgical swabs, and the like that are used for the absorption of fluid therefrom, to aid in wound healing, or for the delivery of active materials, such as medicaments, or moisture.

The term “biodegradable” as used herein refers to the process in which an organic material is exposed to aerobic conditions, the material will break down into simple compounds such as carbon dioxide and water or, under anaerobic conditions, the material will break down into simple compounds such as carbon dioxide, water, and methane by the action of natural occurring microorganisms. Biodegradability means that the organic constituents of the molded or extruded articles are subject to decomposition via biological activity and there is an absence of persistent metabolites. A variety of different standardized biodegradability methods have been established by various organizations and in different countries. For example, for aerobic biodegradability, the American Society for Testing and Materials (ASTM) has established ASTM D 5338 (Standard Test Method for the Determining Aerobic Biodegradation of Plastic Materials Under Controlled Composting Conditions) for municipal solid waste composting, and ASTM D 5271 (Standard Test Method for Assessing the Aerobic Biodegradation of Plastic Materials in an Activated Sludge Wastewater Treatment System) for municipal wastewater treatment. These tests measure the percent of test material that mineralizes as a function of time by monitoring the amount of carbon dioxide being released as a result of assimilation by microorganisms in the matrix of interest. The carbon dioxide production in these tests is typically measured via electrolytic respirometry. Other standard protocols, such 301B from the Organization for Economic Cooperation and Development (OECD), may also be used to assess the aerobic biodegradability of a material. Standard biodegradation tests in the absence of oxygen are described in various protocols such as ASTM D 5511 (Standard Test Method for Determining the Anaerobic Biodegradation of Plastic Materials Under High Solids Anaerobic Digestion Conditions) or ASTM D 5526 (Standard Test Method for Determining Anaerobic Biodegradation of Plastic Materials Under Accelerated Landfill Conditions). These tests are used to assess the biodegradability of materials in septic tanks, anaerobic digestion or sanitary landfills.

“Flushable” as used herein means that an article can be safely flushed into a sewer system without detrimental consequences to existing sewage infrastructure systems.

The term “hygienic articles” as used herein includes absorbent articles, wet wipes and the like used for maintenance of a bodily hygiene.

The term “interlabial pad,” refers to an absorbent article worn in the interlabial space for the absorption of fluid and/or gas therefrom, to aid in wound healing, or for the delivery of active materials, such as medicaments, or moisture. The interlabial pad comprises a liquid pervious topsheet, liquid impervious backsheet and an absorbent core disposed between the topsheet and the backsheet. Examples of such devices are described in U.S. Pat. No. 2,917,049 issued to Delaney on Dec. 15, 1959, U.S. Pat. No. 3,420,235 issued to Harmon on Jan. 7, 1969, U.S. Pat. No. 4,595,392 issued to Johnson, et al. on Jun. 17, 1986, and U.S. Pat. No. 5,484,429 issued to Vukos, et al. on Jan. 16, 1996. A commercially available interlabial device is the INSYNC Miniform interlabial pad which is marketed by A-Fem of Portland, Oreg. and described in U.S. Pat. Nos. 3,983,873 and 4,175,561 issued to Hirschman on Oct. 5, 1976 and Nov. 27, 1979, respectively.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

The term “laminate” as used herein refers to one or more layers of material.

The term “nonwoven,” as used herein, refers to a web having a structure of individual fibers or threads, which are interlaid, but not in a regular, repeating manner as in a woven or knitted fabric. Nonwoven webs or fabrics have been formed from many processes, such as, for example, meltblowing processes, spunbonding processes, hydroentangling processes, bonded carded web processes, and wet laid web making processes, such as paper making from wood pulp and cellolusic fibers or the combination of air laid and wet laid. The basis weight of nonwoven fabrics is usually expressed in grams per square meter and the fiber diameters are usually expressed in microns. Fiber size can also be expressed in denier. The basis weight of nonwoven webs useful as components of the present invention, such as the facing layer (which can be a single layer or a composite of more than one layer), can range from 10 to 200 g/m². The constituent fibers of nonwoven webs can be wood pulp, cellulosic fibers, or other polymer fibers, which may include can be monocomponent, bicomponent and/or biconstituent, capillary channel fibers, having a major cross-sectional dimension (e.g., diameter for round fibers) ranging from 5-200 microns. The constituent fibers can range from about 0.1 denier to about 100 denier.

As used herein, the terms “pantiliner,” and “sanitary napkin,” refers to absorbent articles worn external about the pudenal region for the absorption of fluid therefrom, to aid in wound healing, or for the delivery of active materials, such as medicaments, or moisture. Sanitary napkins typically comprise a liquid pervious topsheet, liquid impervious backsheet and an absorbent core disposed between the topsheet and the backsheet. The sanitary napkin, as well as each layer or component thereof can be described as having a “body facing” surface and a “garment facing” surface. Pantiliners and sanitary napkins may have side extensions commonly referred to as “wings,” designed to wrap the sides of the crotch region of the panties of the user of the sanitary napkin that may be an extension of the topsheet and/or the backsheet. Such devices are disclosed in U.S. Pat. No. 4,463,045 issued to Ahr et al., U.S. Pat. No. 4,556,146 issued to Swanson et al., U.S. Pat. No. 4,950,264 issued to Osborn III, et al. and U.S. Pat. No. 4,687,478 issued to Van Tillburg.

As used herein, the term “polymer” generally includes, but is not limited to, homopolymers, copolymers, (such as for example, block, graft, random and alternating copolymers, terpolymers, etc.,) and blends and modifications thereof. In addition, unless otherwise specifically limited, the term “polymer” includes all possible geometric configurations of the material. The configurations include, but are not limited to, isotactic, atactic, syndiotactic, and random symmetries.

The term “Poly (3-hydroxybutyrate-co-3-hydroxyhexanoate) Copolymers” or “C₄C₆ PHAs” as used herein refers to the polyhydroxyalkanoate copolymers (PHAs) comprising at least two randomly repeating monomer units (referred to also as “RRMUs”)

The polyhydroxyalkanoate copolymer of the present invention comprises at least two randomly repeating monomer units
wherein a first monomer unit has structure (I) 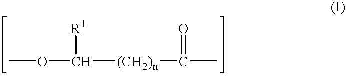
where R¹ is CH₃, and n is 1; and

wherein a second monomer unit has structure (II) 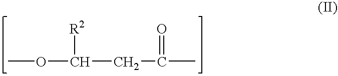
where R² is a CH₂CH₂CH₃

The composition of the present invention may have between 6 and 18 mole percentage of the RRMU's having the structure of the second monomer unit (II). Alternatively, the composition may have between 8 and 16 mole percentage, and between 8 and 15 percentage of the RMMU's have the structure of the second monomer unit (II). Suitably, the molar ratio of the first RRMU to the second RRMU in the copolymer is in the range between 94:6 to 82:18, from about 92:8 to about 84:16, and from about 92:8 to about 85:15. In addition, the polyhydroxyalkanoate copolymer suitably has a number average molecular weight of greater than about 20,000 g/mole, greater than 30,000 g/mole or, in a further embodiment, greater than 50,000 g/mole.

The C₄C₆ PHAs set forth herein can be synthesized by chemical or biological methods as disclosed in, for example, U.S. Pat. No. 5,990,271, issued to Noda on Nov. 23, 1999 and U.S. Pat. No. 5,942,597, issued to Noda, et al. on Aug. 24, 1999 both of which are incorporated herein by reference, Fukui, T. and Doi, Y. Appl. Microbiol. Biotechnol, 49:333-336 (1998), and Kichise, T. et al. Int'l. J. of Biological Macromolecules, 25:69-77 (1999). The amount of C6 in the final product is determined by standard methods such as NMR or GC MS methods such as described in Doi, Y. et al., Macromolecules 28, 4822 (1995) and Fukui, T. et al., Biomacromolecules 3, 618 (2002).

The laminate of the present invention has two components, a C₄C₆ PHA copolymer and a nonwoven.

The C₄C₆ PHAs used in the laminate of the present invention has at least two RRMUs. The first monomer unit has a structure (1), where R¹ is CH₃, and n is 1.

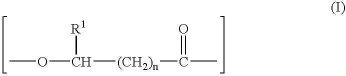

The second monomer unit has structure (II) where R² is a CH₂CH₂CH₃.

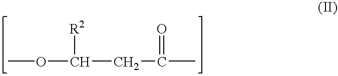

For the present invention, between 6 and 18% of the randomly repeating monomer units have the structure of the second monomer unit (II). Alternatively, the composition may have between 8 and 16 mole percentage, and between 8 and 15 percentage of the RMMU's have the structure of the second monomer unit (II). Further, such compositions are demonstrated herein to provide a bigger processing window and soft and flexible physical properties. A C₄C₆ PHA having this composition crystallizes slowly.

The nonwoven comprised in the laminate of the present invention may be a biodegradable or water dispersible nonwoven.

The resultant laminate of the present invention may be used as a flushable, fluid impervious, absorbent backsheet that provides good stability in use and when stored and shipped. The resultant laminate disintegrates after being flushed down the toilet.

Not to be bound by theory, the inventors believe that the higher the percentage of the monomer II, the slower the copolymer will harden after heat processing. Moreover, the inventors believe that the higher the percentage of the monomer II the slower the crystallization rate. Surprisingly, the compositions of C₄C₆ PHAs in the current invention having the higher percentage of the monomer II perform more effectively, as compared with previously described uses for C₄C₆ PHAs comprising items such as molded articles. In molding, it is preferred to have low percentage of the monomer II so that the C₄C₆ PHAs harden quickly after molding for faster processing, as described in U.S. Pat. No. 6,706,942 issued to Zhao, et al on Mar. 16, 2004.

It is believed that the compositions of the present invention provide a bigger processing window and soft and flexible physical properties. Moreover it is believed that the slow crystallization of the C₄C₆ PHA copolymer allows the laminate to be assembled on typical manufacturing equipment for absorbent articles and at typical manufacturing speeds. For the purposes of providing a laminate in the current invention, a slower hardening rate and higher lingering tacticity is preferred, though not so slow that the characteristic problems with PHAs again becomes disadvantageous. Without being bound by theory, it is believed that the above described optima result from the aforementioned balance between stickiness and hardening over time as the C₄C₆ PHA copolymer cools following lamination or adhesion. The slow crystallization of the C₄C₆ PHA makes the C₄C₆ PHA tacky, thus, thus C₄C₆ PHA layer can serve as an adhesive layer for the absorbent product assembling. The material must be sufficiently sticky to form the desired level of sealing with the nonwoven substrate while not remaining sticky in subsequent processing steps, such as for example, capturing the formed laminate in roll form where latent or residual stickiness could result in undesired roll blocking. When making the laminate, the nonwoven prevents the C₄C₆ PHA from sticking to the processing equipment.

Optional materials may be used as processing aids to modify the processability and/or to modify physical properties such as elasticity, tensile strength and modulus of the final product. Other benefits include, but are not limited to, stability including oxidative stability, brightness, color, flexibility, resiliency, workability, and odor control. These optional ingredients may be present in quantities of less than about 70%, from about 0.1% to about 50%, or from about 0.1% to about 40% by weight of the composition.

Plasticizers may be used in the composition to modify the mechanical properties of products formed from the composition. In general, a plasticizer tends to lower the modulus and tensile strength, and to increase the ultimate tensile elongation, impact strength, and tear strength of the polymeric product. The plasticizer may also be used to lower the melting point of the composition to thereby enable melt-processing at lower temperatures and to minimize energy requirements and thermal degradation. These plasticizers are typically not required in order to obtain the advantageous combination of properties discussed above.

Nonlimiting examples of plasticizers may include those selected from the group consisting of a plasticizer selected from the group consisting of dimethyl sebacate, glycerin, triacetin, glycerol, monostearate, sorbitol, erythritol, glucidol, mannitol, sucrose, ethylene glycol, propylene glycol, diethylene glycol, triethylene glycol, diethylene glycol dibenzoate, dipropylene glycol dibenzoate, triethylene glycol caprate-caprylate, butylene glycol, pentamethylene glycol, hexamethylene glycol, diisobutyl adipate, oleic amide, erucic amide, palmitic amide, dimethyl acetamide, dimethyl sulfoxide, methyl pyrrolidone, tetramethylene sulfone, oxa monoacids, oxa diacids, polyoxa diacids, diglycolic acids, triethyl citrate, acetyl triethyl citrate, tri-n-butyl citrate, acetyl tri-n-butyl citrate, acetyl tri-n-hexyl citrate, alkyl lactates, phthalate polyesters, adipate polyesters, glutate polyesters, diisononyl phthalate, diisodecyl phthalate, dihexyl phthalate, alkyl alylether diester adipate, dibutoxyethoxyethyl adipate, and mixtures thereof. Other plasticizers may be used such as those described in U.S. Pat. No. 3,182,036 issued to Baptist, et al. on May 4, 1965 and U.S. Pat. No. 5,231,148 issued Kleinke, et al. on Jul. 27, 1993. Suitable molecular weights are less than about 20,000 g/mol, less than about 5,000 g/mol, or less than about 1,000 g/mol. If present, the amount of plasticizer in the final molded or extruded article composition is from about 0.1% to about 70%, from about 0.5% to about 50% or from about 1% to about 30%.

Nucleating agents are generally used to increase the crystallization rate, reduce the size of crystals, and improve transparency. Nucleating agents can also improve the meltflow and demolding behavior of partly crystalline plastic materials such as thermoplastic polyesters. A second polyhydroxyalkanoate such as polyhydroxybutyrate can act as a nucleating agent for the first polyhydroxyalkanoate and thereby improve the crystallization rate of the first polyhydroxyalkanoate such as disclosed by Autran, et al. WO02/055581 and WO02/50156, each filed Dec. 20, 2001. Further nucleating agents include talc, boron nitride, titanium oxide, micromica, chalk, salts, sorbitol acetal, clay, calcium carbonate, sodium chloride, calcium phosphate, Licomont CaV 102 and Licomont NaV 101 (the calcium and sodium salt, respectively, of montanic acid, i.e., long chain (C28-C32) linear carboxylic acids) both of which are commercially available from the Clariant Corporation (Coventry, R.I.); and Millad 3988 (1,2,3,4-bis-(3,4-dimethylbenzylidene sorbitol) which is commercially available from Milliken Chemical (Inman, S.C.). Nucleating agents commonly constitute from about 0.01% to about 5% of the weight of the molded or extruded articles, when used.

Further optional ingredients include salts, slip agents, crystallization accelerators or retarders, odor masking agents, cross-linking agents, emulsifiers, surfactants, cyclodextrins, lubricants, other processing aids, optical brighteners, antioxidants, flame retardants, dyes, pigments, fillers, proteins and their alkali salts, waxes, tackifying resins, extenders, chitin, chitosan, and mixtures thereof.

A filler may further be selected from the group of clays, silica, mica, wollastonite, calcium hydroxide, calcium carbonate, sodium carbonate, magnesium carbonate, barium sulfate, magnesium sulfate, kaolin, calcium oxide, magnesium oxide, aluminum hydroxide, talc, titanium dioxide, wood flour, walnut shell flour, alpha cellulose floc, cellulose fibers, chitin, chitosan powders, organosilicone powders, nylon powders, polyester powders, polypropylene powders, starches, and mixtures thereof. When used, the amount of filler is from 0.1% to 60% by weight of the molded or extruded article.

A lubricant may, for example, be selected from the group consisting of metal soaps, hydrocarbon waxes, fatty acids, long-chain alcohols, fatty acid esters, fatty acid amides, silicones, fluorochemicals, acrylics, and mixtures thereof. When used, the amount of lubricant is from 0.1% to 20% by weight of the molded or extruded article.

Natural polymers may also be used in the present invention such as, chitin, cellulose, starch, proteins and the like. Suitable protein-based polymers include soy protein. The natural polymer may be present in an amount of from about 0.1% to about 80% or from about 1% to about 60%.

The laminate is formed during the manufacturing of the absorbent article by melt processing, such as melt extrusion or hot melt spraying, the polyhydroxyalkanoate copolymer on a nonwoven to form a uniform layer.

The nonwovens used to make the laminate of the present invention may be biodegradable nonwovens and water dispersible nonwovens. Some examples of biodegradable nonwovens that may be used making the laminate of the present included nonwovens comprised of fabric materials selected from the group consisting of rayon, cotton, wood pulp, starch, polyvinyl alcohol, polyethylene oxide and mixtures thereof. The term “water-dispersible nonwovens,” as used herein are nonwovens comprised of water-dispersible binders that may disperse into loose fibers under large amount of water. The binders are selected from the group consisting of water dispersible starch, polyvinyl alcohol, water dispersible polyesters and polyacrylates. The fibers that comprise the water dispersible nonwovens are selected from the group consisting of polyolefin, polyethylene, polypropylene, polyester, polyamide, rayon, cotton, wood pulp, starch, aliphatic polyester, aliphatic-aromatic polyester, polyester amide, polyvinyl alcohol, polyethylene oxide and mixtures thereof.

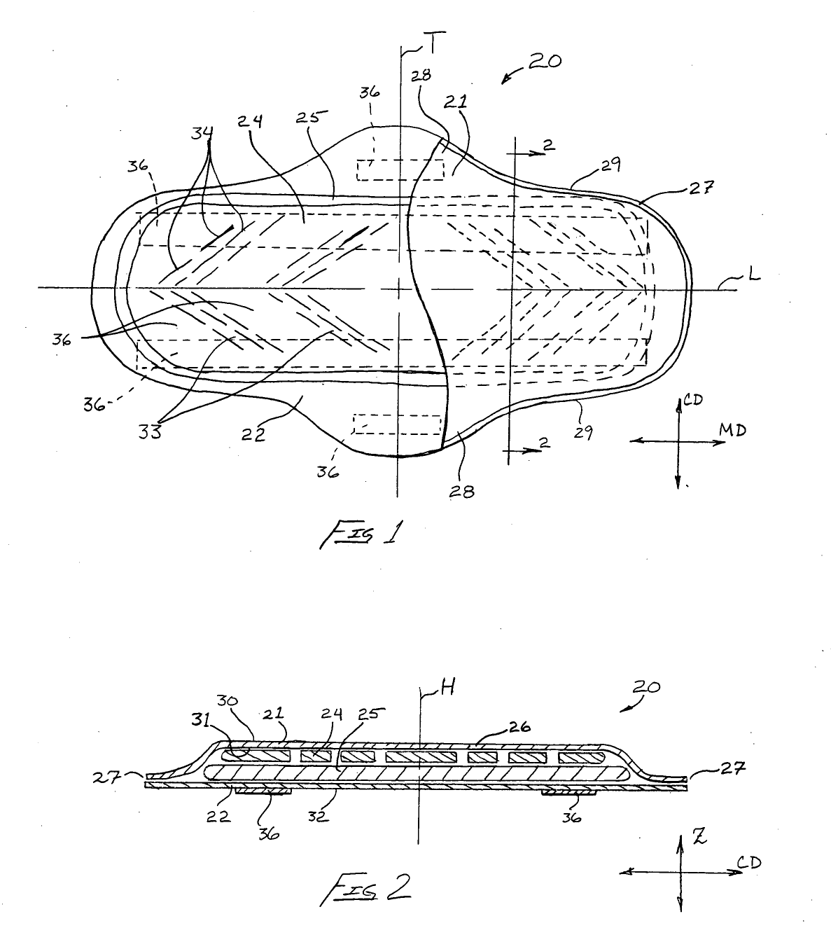

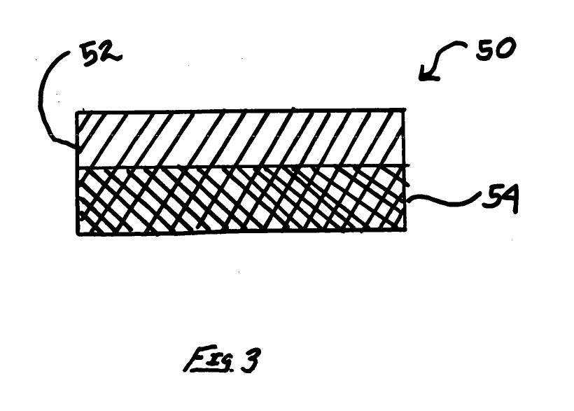

FIG. 1 and FIG. 2 show one embodiment of an absorbent article of the present invention. FIG. 1 and FIG. 2 show a sanitary napkin 20 comprising a flushable, fluid permeable facing layer 21, a flushable fluid impervious backsheet 22, and an flushable, absorbent core 24 joined to the facing layer 21. The facing layer 21 and backsheet 22 can be joined about a periphery 27. Facing layer 21 of sanitary napkin 20 can be a body-contacting layer commonly known in the art as a topsheet 26. Facing layer 21 can be a composite comprising a topsheet 26 and a second absorbent layer, not shown in FIG. 1 or FIG. 2, but also known in the art. An optional second absorbent layer 25 can be disposed between absorbent core 24 and breathable backsheet 22.

The sanitary napkin 20, as well as each layer or component thereof can be described as having a “body facing” surface and a “garment facing” surface. As can be readily understood by considering the ultimate use for sanitary napkins, the body facing surfaces are the surfaces of the layers or components that are oriented closer to the body of the user when in use, and the garment facing surfaces are the surfaces that are oriented closer to the undergarment of the user when in use. Therefore, for example, facing layer 21 has a body facing surface 30 and a garment facing surface 31 that is the surface that can be adhered to the underlying first absorbent layer 24. The garment facing surface 32 of the backsheet 22 of a sanitary napkin, for example, is oriented closest to and may contact the wearer's panties in use (via adhesive attachment means, if used).

Sanitary napkin 20 can have side extensions 28, commonly referred to as “wings,” designed to wrap the sides of the crotch region of the panties of the user of sanitary napkin 20. Sanitary napkin 20 and/or wings 28 can have fastening means including attachment components, such as pressure sensitive adhesive, or mechanical fasteners such as hook and loop fasteners. The sanitary napkin 20 shown in FIG. 1 and FIG. 2 has strips of positioning adhesive 36 on the garment-facing surface 32 of backsheet 22. The positioning adhesive 36 can be a flushable hot-melt adhesive material capable of establishing a temporary bond with the undergarment material. A suitable material is the composition designated HL-1491 XZP, commercially available from H. B. Fuller, Toronto, Ontario, Canada. The fastening means can include attachment components such as positioning adhesive 36, disposed on the extensions 28, as shown in FIG. 1 and FIG. 2. The positioning adhesive 36, or other attachment components can be applied to the garment facing surface 32 of the backsheet 22 in various patterns including a complete adhesive coverage, parallel longitudinal strips or lines, a line of adhesive following the perimeter of the structure, transverse lines of adhesive, and the like, or any combination thereof.

In general, the presence of fastening member defines an attachment zone that is coextensive with the fastening means. The attachment zone is the portion of the sanitary napkin, typically the backsheet thereof, that is fixed with respect to the wearer's panties. The sanitary napkin 20 can be made by hand or on commercial high-speed production lines, as is known in the art.

Facing layer 21 may, in some embodiments, comprise flushable nonwoven materials or flushable apertured polymer films. Extensions 28, if used, can be integral extensions of the facing layer or the backsheet or both, and they can be symmetric about the longitudinal axis L, transverse axis T, or both. The facing layer 21 may comprise biodegradable nonwovens and water dispersible nonwovens. Some examples of biodegradable nonwovens include nonwovens comprised of fabric materials selected from the group consisting of rayon, cotton, wood pulp, starch, polyvinyl alcohol, polyethylene oxide and mixtures thereof. The term “water-dispersible nonwovens,” as used herein are nonwovens comprised of water-dispersible binders that may disperse into loose fibers under large amount of water. The binders are selected from the group consisting of water dispersible starch, polyvinyl alcohol, water dispersible polyesters and polyacrylates. The fibers that comprise the water dispersible nonwovens are selected from the group consisting of polyolefin, polyethylene, polypropylene, polyester, polyamide, rayon, cotton, wood pulp, starch, aliphatic polyester, aliphatic-aromatic polyester, polyester amide, polyvinyl alcohol, polyethylene oxide and mixtures thereof. Nonwoven webs may be composites of two or more nonwoven webs.

The absorbent core 24 (and second absorbent layer 25, if used) and backsheet 22 can comprise flushable absorbent materials, and liquid impermeable film materials respectively, as is well known in the art. The flushable absorbent core 24 can be Foley Fluff pulp (available from Buckeye Technologies Inc., Memphis, Tenn.) that is disintegrated and formed into a core having a density of about 0.07 grams per cubic centimeter (g/cm³) and a caliper of about 10 mm.

The absorbent article according to the present invention also comprises a flushable backsheet 22. The backsheet 22 prevents the exudates absorbed and contained in the absorbent structure from wetting the articles that contact the absorbent product, such as underpants, pants, pajamas, undergarments, and shirts or jackets, thereby acting as a barrier to fluid transport. In addition, however, the backsheet 22 of the present invention permits the transfer of at least water vapor, typically both water vapor and air, through the backsheet 22, and thus allows the circulation of air into and water vapor out of the article. The backsheet 22 typically extends across the whole of the absorbent structure and can also extend into and form part or all of extensions 28, sideflaps, and side wrapping elements or wings, if present.

The flushable backsheet 22 may be a biodegradable nonwoven or water dispersible nonwoven. The backsheet 22 is formed during the manufacturing of the absorbent article by melt processing, such as melt extrusion or hot melt spraying, of a C₄C₆ PHA copolymer on the nonwoven to form a uniform layer. While the copolymer is still not crystallized and remains tacky, the absorbent core 24 is laid on top of the C₄C₆ PHA copolymer layer. Then, the topsheet 26 is laid on top of the absorbent core 24. The absorbent core 24 is sandwiched by the topsheet 26 and backsheet 22. Finally the topsheet 26 and backsheet 22 are sealed together by any means known in the art, such as by adhesive bonding, thermal bonding, ultrasonic bonding, and the like. Complete bonding at the interface is not necessary in some embodiments. One such adhesive that may be used is a meltblown thermoplastic adhesive or a fluid permeable adhesive, such as the aforementioned Findley HX1500-1 adhesive. Adhesion can be provided by application of a substantially uniform layer of adhesive applied by means known in the art, such as by spraying or slot coating.

EXAMPLES OF THE PRESENT INVENTION

| | | | Laminate Backsheet | |
|---|---|---|---|---|
| | Topsheet | Absorbent Core | Layer facing core | Outer layer |
| Sample 1 | apertured rayon | wood pulp, | poly(hydroxybutyrate- | BOUNTY paper |
| | nonwoven hydro- | 55 gsm | co-hexanoate), 10% | towel, 30 gsm |
| | entangled, | | hexanoate | |
| | 33 gsm | | | |
| Sample 2 | apertured rayon | wood pulp, | poly(hydroxybutyrate- | non-apertured |
| | nonwoven hydro- | 55 gsm | co-hexanoate), 10% | rayon nonwoven, |
| | entangled, | | hexanoate | hydro-entangled, |
| | 33 gsm | | | 33 gsm |
| Sample 3 | apertured rayon | wood pulp, | poly(hydroxybutyrate- | non-apertured |
| | nonwoven hydro- | 55 gsm | co-hexanoate), 10% | cotton nonwoven |
| | entangled, | | hexanoate | hydro-entangled, |
| | 33 gsm | | | 40 gsm |
| Sample 4 | apertured rayon | wood pulp, | poly(hydroxybutyrate- | rayon, carded w |
| | nonwoven, | 55 gsm | co-hexanoate), 10% | starch binder, 15 |
| | hydro-entangled, | | hexanoate | gsm |
| | 33 gsm | | | |
| Sample 5 | apertured rayon | wood pulp, | poly(hydroxybutyrate- | tissue paper |
| | nonwoven, | 55 gsm | co-hexanoate), 10% | |
| | hydro-entangled, | | hexanoate | |
| | 33 gsm | | | |
| Sample 6 | Non-apertured | wood pulp, | poly(hydroxybutyrate- | tissue paper |
| | rayon nonwoven, | 55 gsm | co-hexanoate), 10% | |
| | hydro-entangled, | | hexanoate | |
| | 33 gsm | | | |

FIG. 3 shows a cross sectional view of the laminate 50 of the present invention. The laminate 50 of the present invention has two components, a C₄C₆ PHA copolymer 52 and a nonwoven 54. The C₄C₆ PHA copolymer 52 is the top layer of the laminate 50. The nonwoven 54 is the bottom layer of the laminate 50. The C₄C₆ PHA 52 services as an adhesive layer for the absorbent product assembly, as described above.

### Test Methods

The samples consist of the following C₄C₆ PHA formulas: (A) 4% C₆ and 96% C₄, (B) 6% C₆ and 94% C₄, (C) 8% C₆ and 92% C₄, (D) 10% C₆ and 90% C₄ and (E) 15% C₆ and 85% C₄. The size of the sample ranges from about 10 mg to about 20 mg.

### I. Melting Temperature

The graph shows the effect temperature has on various compositions of C₄C₆ PHAs. The melting temperature of each sample was measured on a Differential Scanning Calorimeter, manufactured by PerkinElmer, Inc of Boston, Mass. USA. The sample is heated at a rate of 20° C./min. The melting temperature (Tm) is the highest temperature of the endothermic peak on the graph below.

[image]

[image]

The graph above illustrates the loss in molecular weight that can occur during a combination of compounding and film extrusion. The starting molecular weight M_{wo} is determined prior to the melt process and M_{w} is the molecular weight determined after melt processing. The two shear rates represent the approximate upper and lower bounds imposed on the polymer melt in a typical extruder. The two total residence times bracket approximately the average time the polymer melt is exposed to the high temperature/high shear environment. The rpm is the extruder motor speed. The graph shows that process temperatures below about 160° C. are preferred so as to minimize degradation. This upper process temperature is called melt degradation temperature of C₄C₆ PHAs, where the C₄C₆ PHA copolymers start losing molecular weight significantly.

The above graphs show that the lower the percentage of C₆ in the C₄C₆ PHA composition, the closer the melt temperature is to the decomposition temperature for that composition. Conversely, the graphs show that the higher the percentage of C₆ in the C₄C₆ PHA composition the farther the melt temperature is from the decomposition temperature.

### II. Crystallization Kinetics

Crystallization kinetics of the samples were measured on a Differential Scanning Calorimeter, manufactured by PerkinElmer, Inc of Boston, Mass. USA. Each of the samples is heated isothermally for 1 minute at 160° C. Then, the temperature of the sample is quenched to 40° C. and is maintained at a temperature at 40° C. The crystallization time for each sample is measured as the shortest time duration to produce an exothermic peak during isothermal heating at 40° C.

[image]

The above graph shows that the lower percentage of C₆ in the C₄C₆ PHA composition the faster the crystallization. Conversely, the higher the percentage of C_{6 in the C}₄C₆ PHA composition the slower the crystallization.

One of the goals of the present invention is find a C₄C₆ PHA composition which may be used in manufacturing a laminate and backsheet in an aborbent article and can be produced at typical manufacturing speed, on typical manufacturing equipment by melt processing. The above test methods assess the effect of composition of C₄C₆ PHA on melting temperature and crystallization kinetics.

The results of the test methods reveal two trends. The first trend shown is that the lower the percentage of C₆ in the C₄C₆ PHA composition, the closer the melting temperature is to the decomposition temperature for that composition. The second trend that is shown is the lower percentage of C₆ in the C₄C₆ PHA composition the faster the crystallization. Due to the closeness of the melt temperature to the decomposition crystallization temperature, C₄C₆ PHA's with lower percentage of C₆, such as 4% C₆, have narrow processing windows. Therefore, C₄C₆ PHA compositions with a lower percentage of C₆ are not typically used for melt processing. Conversely, the higher the percentage of C₆ in the C₄C₆ PHA composition, the farther away the melt temperature is to the decompostion temperature for that composition, however, such composition have slower crystallization. The slow crystallization means that composition with higher percentages of C₆ remain tacky longer. Therefore, composition of C₄C₆ PHA with higher percentages of C₆ may not be used in melt processing because these composition remain tacky for longer periods of time and tend to stick on processing equipment slowing down production. Hence a C₄C₆ PHA composition with a 6 to 18% or 8 to 16% or 8 to 15% of C₆ are used for the purposes of the present invention because the composition does not decompose at melting temperature and does not remain tacky overly long during processing.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A laminate comprising:
a polyhydroxyalkanoate copolymer comprising at least two randomly repeating monomer units
wherein a first monomer unit has structure (I) 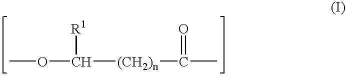
where R¹ is CH₃, and n is 1; and
wherein a second monomer unit has structure (II) 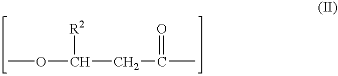
where R² is a CH₂CH₂CH₃wherein between 6 and 18% of the randomly repeating monomer units has the structure of the second monomer unit, and a nonwoven.

2. The laminate of claim 1 wherein between 8 and 16% of the randomly repeating monomer units have the structure of the second monomer unit.

3. The laminate of claim 1 wherein said nonwoven is a water-dispersible nonwoven selected from the group consisting of polyolefin, polyethylene, polypropylene, polyester, polyamide, rayon, cotton, wood pulp, starch, aliphatic polyester, aliphatic-aromatic polyester, polyester amide, polyvinyl alcohol, polyethylene oxide and mixtures thereof.

4. The laminate of claim 1 wherein said nonwoven is a biodegradable nonwoven selected from the group consisting of rayon, cotton, wood pulp, starch, polyvinyl alcohol, polyethylene oxide and mixtures thereof.

5. The laminate of claim 1 in the form of fluid impervious backsheet of an absorbent article.

6. The laminate of claim 1 further comprising a processing aid.

7. The laminate of claim 1 wherein the processing aid is a plasticizer selected from the group consisting of dimethyl sebacate, glycerin, triacetin, glycerol, monostearate, sorbitol, erythritol, glucidol, mannitol, sucrose, ethylene glycol, propylene glycol, diethylene glycol, triethylene glycol, diethylene glycol dibenzoate, dipropylene glycol dibenzoate, triethylene glycol caprate-caprylate, butylene glycol, pentamethylene glycol, hexamethylene glycol, diisobutyl adipate, oleic amide, erucic amide, palmitic amide, dimethyl acetamide, dimethyl sulfoxide, methyl pyrrolidone, tetramethylene sulfone, oxa monoacids, oxa diacids, polyoxa diacids, diglycolic acids, triethyl citrate, acetyl triethyl citrate, tri-n-butyl citrate, acetyl tri-n-butyl citrate, acetyl tri-n-hexyl citrate, alkyl lactates, phthalate polyesters, adipate polyesters, glutate polyesters, diisononyl phthalate, diisodecyl phthalate, dihexyl phthalate, alkyl alylether diester adipate, dibutoxyethoxyethyl adipate, and mixtures thereof.

8. A flushable absorbent articles comprising:
a) a fluid permeable topsheet
b) an absorbent core
c) a flushable fluid impervious backsheet comprising:
a polyhydroxyalkanoate copolymer comprising at least two randomly repeating monomer units
wherein a first monomer unit has structure (I) 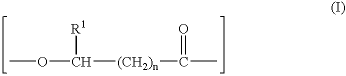
where R¹ is CH₃, and n is 1; and
wherein a second monomer unit has structure (II) 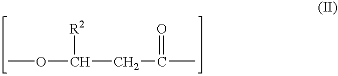
wherein R² is a CH₂CH₂CH₃:
wherein between 6 and 18% of the randomly repeating monomer units has the structure of the second monomer unit, and a nonwoven.

9. The flushable absorbent article of claim 8 is a flushable feminine pad.

10. The flushable absorbent article of claim 8 is a flushable pantiliner.

11. The flushable absorbent article of claim 8 is a flushable inner labia pad.

12. The flushable fluid impervious backsheet of claim 8 wherein between 8 and 16% of the randomly repeating monomer units has the structure of the second monomer unit.

13. The flushable fluid impervious backsheet of claim 8 wherein said nonwoven is a biodegradable nonwoven selected from the group consisting of rayon, cotton, wood pulp, starch, polyvinyl alcohol, polyethylene oxide and mixtures thereof.

14. The flushable fluid impervious backsheet of claim 8 wherein said nonwoven is a water-dispersible nonwoven selected from the group consisting of polyolefin, polyethylene, polypropylene, polyester, polyamide, rayon, cotton, wood pulp, starch, aliphatic polyester, aliphatic-aromatic polyester, polyester amide, polyvinyl alcohol, polyethylene oxide and mixtures thereof.

15. The flushable fluid impervious backsheet of claim 8 wherein the processing aid is a plasticizer selected from the group consisting of dimethyl sebacate, glycerin, triacetin, glycerol, monostearate, sorbitol, erythritol, glucidol, mannitol, sucrose, ethylene glycol, propylene glycol, diethylene glycol, triethylene glycol, diethylene glycol dibenzoate, dipropylene glycol dibenzoate, triethylene glycol caprate-caprylate, butylene glycol, pentamethylene glycol, hexamethylene glycol, diisobutyl adipate, oleic amide, erucic amide, palmitic amide, dimethyl acetamide, dimethyl sulfoxide, methyl pyrrolidone, tetramethylene sulfone, oxa monoacids, oxa diacids, polyoxa diacids, diglycolic acids, triethyl citrate, acetyl triethyl citrate, tri-n-butyl citrate, acetyl tri-n-butyl citrate, acetyl tri-n-hexyl citrate, alkyl lactates, phthalate polyesters, adipate polyesters, glutate polyesters, diisononyl phthalate, diisodecyl phthalate, dihexyl phthalate, alkyl alylether diester adipate, dibutoxyethoxyethyl adipate, and mixtures thereof.
